(19) United States
(12) Patent Application Publication
(10) Pub. No.: US 2011/0230942 A1
(43) Pub. Date: Sep. 22, 2011

(54) HIGH-RESOLUTION INFRARED IMAGING FOR ENHANCED DETECTION, DIAGNOSIS, AND TREATMENT OF CUTANEOUS LESIONS

(75) Inventors: Cila Herman, Towson, MD (US); Rhoda Alani, Baltimore, MD (US); Muge Pirtini Cetingul, Baltimore, MD (US)

(73) Assignee: The Johns Hopkins University, Baltimore, MD (US)

(21) Appl. No.: 13/129,764

(22) PCT Filed: Jun. 1, 2009
(86) PCT No.: PCT/US09/03319

§ 371 Date: May 17, 2011

Related U.S. Application Data
(60) Provisional application No. 61/118,783, filed on Dec. 1, 2008.
(60) Provisional application No. 61/122,770, filed on Dec. 16, 2008.

(51) Int. Cl. A61F 7/00

(52) U.S. Cl. 607/96

(57) ABSTRACT

A medical diagnosis system, comprising: a thermal stimulator; an infrared detection system constructed and arranged to detect infrared radiation from at least a portion of a subject under observation to provide an output signal from the portion of the subject after undergoing thermal stimulation from said thermal stimulator; and a signal processor in communication with the infrared detection system to receive the output signal from the infrared detection system, wherein the signal processor determines a measured thermal response of the portion of the subject to the thermal stimulation and compares the measured thermal response to an expected thermal response to determine a presence of an abnormality.

### CROSS-REFERENCE TO RELATED APPLICATIONS

This application claims priority to U.S. Provisional Application No. 61/118,783 filed Dec. 1, 2008 and U.S. Provisional Application No. 61/122,770 filed Dec. 16, 2008, the entire contents of which are hereby incorporated by reference.

The U.S. Government has a paid-up license in this invention and the right in limited circumstances to require the patent owner to license others on reasonable terms as provided for by the terms of Grant No.: 0651981, awarded by the National Science Foundation.

### BACKGROUND

1. Field of Invention

The current invention relates to medical diagnosis, and more particularly to medical diagnosis of lesions using infrared imaging.

2. Discussion of Related Art

Melanoma incidence is increasing at one of the fastest rates for all cancers in the United States with a current lifetime risk of 1 in 58. Over 60,000 patients are expected to be diagnosed with melanoma in the US with more than 8,000 deaths in 2008. The reported 1-year survival rates for patients with advanced melanoma range from 40% to 60%, and systemic agents are not currently available to significantly extend the lifespan of patients with advanced disease. These statistics stress the need to detect melanomas at their earliest stages for chances of optimal cure and to identify patients with high-risk primary disease for the initiation of early prophylactic treatment.

The increased availability of thermal imaging cameras has led to a growing interest in the application of infrared imaging techniques to the detection and identification of subsurface structures both in engineering and in living systems. Infrared (IR) imaging is a non-contact sensing method concerned with the measurement of electromagnetic radiation in the infrared region of the spectrum (750 nm-100 μm). Radiation emitted by a surface at a given temperature is called spectral radiance and is defined by the Planck's distribution for the idealized case of a blackbody. Infrared cameras detect this radiation and the surface temperature distribution can be recovered after post-processing the sensor information and appropriate calibration. Since the surface temperature distribution depends on the properties of subsurface structures and regions, infrared imaging can be used to detect and identify subsurface structures by analyzing the differences in the thermal response of an undisturbed region such as healthy skin and a near-surface structure of different properties such as a skin lesion.

Infrared imaging can be performed either passively or actively (dynamically). Passive infrared imaging involves, in its simples form, the visualization of the emitted radiation in the infrared region of the electromagnetic spectrum, for example night vision goggles, and, in more advanced imaging applications, measuring (after post processing of the information acquired by the sensor and appropriate calibration) temperature variations of structures whose temperature naturally differs from ambient temperature or varies locally due to internal heat sources. Active infrared imaging involves introducing external forcing such as heating or cooling to induce and/or enhance relevant thermal contrasts observed on the surface. The latter technique is based on the following principle: when a surface is heated or cooled, variations in the thermal properties of a structure located underneath the surface result in identifiable temperature contours on the surface itself, differing from those present in the steady-state situation during passive imaging as well as from the surrounding regions. These contours are characteristic of the thermal properties of the base structure and subsurface perturbations, and can, when combined with a suitable model, provide information regarding the shape and depth of the perturbation (a lesion in our study). Thus, the dynamic thermal response of the structure obtained using the active imaging provides additional information useful in the identification of the perturbation when compared to information obtained by passive imaging.

Infrared imaging has been successfully applied in various problems in engineering and medicine. Recent improvements in infrared sensor and computer technology led to the resurgence of infrared imaging in medicine. In particular, describing the thermal response of chemically and metabolically active multilayered samples constitutes an important problem. For instance, thermal modeling of temperature distributions linked to large blood vessels has received a great deal of attention in the research community (Hundhausen, E and Theves B 1979 Eur. J. Appl. Physiol. Occup. Physiol. 40(4) 235-44; Lemons D E, Chien S, Crawshaw L I, Weinbaum S and Jiji L M 1987, Am. J. Physiol. 253 128-35; Nitzan M, Mahler Y, Roberts J, Khan O, Gluck E, Roberts V C and Baum M 1989, Clin. Phys. Physiol. Meas. 10 337-41; Zhu L and Weinbaum S 1995, J. Biomech. Eng. 117 64-73; Nakagawa A, T. Hirano, Uenohara H, Sato M, Kusaka Y, Shirane R, Takayama K and Yoshimoto T 2003, Minim. Invasive Neurosurg. 46(4) 231). Shrivastava et al (Shrivastava D, McKay B and Roemer R B 2005, J. Heat Trans. 127 179-88) derived an analytical model describing the tissue temperature distribution in unheated/heated, finite, noninsulated tissue with a pair of vessels to quantify the vessel-vessel and vessel-tissue heat transfer rate. He et al (He Y, Liu H, Himeno R, Sunaga J, Kakusho H and Yokota H 2008, Comp. Bio. Med. 38 555-62) developed a FEM model based on the heat transport in porous media to simulate blood flow in large vessels and living tissue. Boue et al (Boue C, Cassagne F, Massoud C and Fournier D 2007, Infrared Phys. Tech. 51 13-20) analyzed the infrared images to extract the radius, depth and the blood flow velocity in a vein. However, the problem of quantifying such response from healthy skin tissue or from melanoma lesions still remains unresolved.

In order to understand the physics of IR imaging in the analysis of lesions, first, it is worth recalling that the chemical reactions, blood transport, perfusion and metabolic processes that affect local temperature response in normal tissue are under both global and local control (Gulyaev Y V Markov A G Koreneva L G and Zakharov P V 1995, IEEE Eng. Med. Bio. 14(6) 766-71; Jones B F and Plassmann P 2002, IEEE Eng. Med. Bio. 21(6) 41-48; Otsuka K, Okada S, Hassan M and Togawa T 2002, IEEE Eng. Med. Bio. 21(6) 49-55; Kakuta N, Yokoyama S and Mabuchi K 2002, IEEE Eng. Med. Bio. 21(6) 65-72). When a cancerous lesion develops, affected tissue has escaped from the control of the various feedback systems and mechanisms present in healthy tissue, leading to such abnormal processes as cell proliferation, disordered spatial organization and excess metabolism i.e. heat generation (Brown S L, Hunt J W and Hill R P 1992, Int. J. Hyperthermia 8(4) 501-14; Jones B F 1998, IEEE Trans. Med. Imaging 17(6) 1019-27). Examples of such a response include Kaposi Sarcoma, melanoma, neuroblastoma, wine stain birthmarks, breast cancer, etc. (Ahuja A S, Prasad K N, Hendee W R and Carson P L 1978, Med. Phys. 5(5) 418-21; Anvari B, Tanenbaum B S, Milner T E, Kimel S, Svaasand L O and Nelson J S 1995, Phys. Med. Biol. 40(9) 1451-65, Head J F and Elliott R L 2002, IEEE Eng. Med. Bio. 21(6) 80-85; Xianwu T, Haishu D, Guangzhi W and Zhongqi L 2004, Proc. 26^{th} IEEE EMBS Ann. Int. Conf. 873-8; Deng Z and Liu J 2005, Proc. 27^{th} IEEE EMBS Ann. Int. Conf. 7525-8; Buzug T M, Schumann S, Pfaffmann L, Reinhold U and Ruhlmann J 2006, Proc. 28^{th} IEEE EMBS Ann. Int. Conf 2766; Mital M and Scott E P 2007, J. Biomech. Eng. 129 33-9).

There is a need in the art to take advantage of these changes and effects visualized by thermal imaging, to distinguish between abnormal and healthy tissue by solving the problem of quantifying such responses from healthy skin tissue and from cutaneous lesions.

### SUMMARY

Some embodiments of the current invention provide a medical diagnosis system, comprising: a thermal stimulator; an infrared detection system constructed and arranged to detect infrared radiation from at least a portion of a subject under observation to provide an output signal from the portion of the subject after undergoing thermal stimulation from said thermal stimulator; and a signal processor in communication with the infrared detection system to receive the output signal from the infrared detection system, wherein the signal processor determines a measured thermal response of the portion of the subject to the thermal stimulation and compares the measured thermal response to an expected thermal response to determine a presence of an abnormality.

Some embodiments of the current invention provide a method of diagnosing a suspected abnormality, comprising: thermally stimulating at least a portion of a subject under observation having the suspected abnormality; detecting infrared radiation to provide an output signal from the at least a portion of the subject after the thermally stimulating; processing the output signal to compare a measured thermal response of the portion of the subject after the thermally stimulating to an expected thermal response to determine a presence of the abnormality.

Some embodiments of the current invention provide a computer readable medium, when executed by a computer, causes the computer to implement the method above.

### BRIEF DESCRIPTION OF THE DRAWINGS

Further objectives and advantages will become apparent from a consideration of the description, drawings, and examples.

FIG. 1 shows a schematic diagram of an embodiment of the invention.

FIG. 2 shows the repeatability of calibrated temperature detected by an infrared detection system.

FIGS. 3a and 3b show an uncorrected temperature map and the corresponding corrected temperature map of human skin, respectively.

FIG. 4 shows a photograph of an experimental set-up and a schematic of the set-up.

FIG. 5a shows the temperature map of a phantom at steady state.

FIG. 5b shows the temperature map of the phantom having undergone a cooling excitation of 120 s.

FIG. 5c-5f show the temperature maps of the phantom during recovery at 30 s, 90 s, 400 s and 720 s after the cooling excitation, respectively.

FIG. 6 shows the temperature profile of the phantom during recovery at 10 s, 20 s, 30 s, 40 s, 50 s, and 60 s after excitation.

FIG. 7 shows a model of a skin lesion.

FIG. 8a shows the cross-sectional temperature map of the model in FIG. 7 during steady state.

FIG. 8b shows the cross-sectional temperature map of the model in FIG. 7 after a 120 s of cooling excitation.

FIG. 8c-8f show the cross-sectional temperature maps of the model in FIG. 7 during recovery at 15 s, 30 s, 45 s, and 60 s after the cooling excitation.

FIG. 9a-9h show the surface temperature maps of the model in FIG. 7 during recovery at 15 s, 30 s, 45 s, and 60 s after the cooling excitation.

FIG. 10 shows the surface temperature profiles for the model in FIG. 7 during recovery.

FIG. 11 shows the temperature differences for varying values of the specific heat of the dermis at different recovery times.

FIG. 12a shows focal points FP1, FP2 and line scratch LS as clearly visible in the infrared image at the start of applying a thermally cooling stimulation.

FIG. 12b shows focal points FP1, FP2 and line scratch LS nearly disappearing 120 s after application.

FIGS. 12c-e show the skin temperature during the thermal recovery phase after 2 s, 20 s and 600 s of thermal stimulation.

FIG. 13a shows temperature profiles measured at different time instants during thermal recovery (each curve corresponds to a time instant from 2 s to 600 s) in a skin cross section encompassing regions of undisturbed tissue UDT and the region of the focal point FP1.

FIG. 13b shows temporal temperature distributions for focal points FP1, FP2, line scratch LS and undisturbed tissue UDT.

FIG. 14 shows a flow chart of another embodiment of the invention.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited herein are incorporated by reference as if each had been individually incorporated.

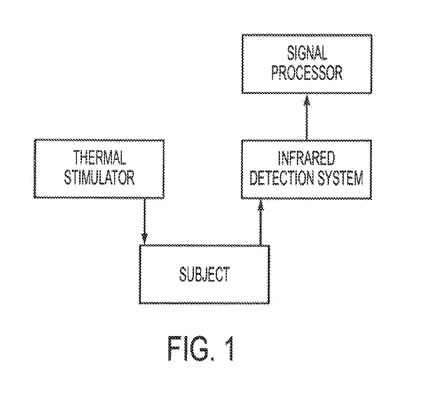

FIG. 1 is a schematic diagram of an embodiment of the invention. A thermal stimulator delivers a thermal stimulation to a subject under observation. The thermal stimulator can deliver a cooling stress by, for example, blowing cold air using a tube. Water, ice or a cold plate can also be used for the cooling stress. The thermal stimulator can deliver a heating stress by, for example, blowing warm air. Water or warm plate can also be used for the heating stress. However, the broad concepts of the current invention are not limited to only blowing warm or cool air for causing a thermal stimulus. The thermal stimulation can be modulated in some embodiments of the current invention. For example, the amplitude of cooling or heating stress can be varied during the thermal stimulation. An infrared detection system is constructed and arranged to detect infrared radiation from at least a portion of a subject under observation to provide an output signal from the at least a portion of the subject having undergone thermal stimulation from the thermal stimulator. The portion of the subject can be an extended external surface region that substantially covers a torso or back (also head, arms legs). The portion of the subject can also be mucosal surfaces along the digestive or respiratory tract. The infrared detection system may comprise, for example, an infrared camera, a confocal microscope, etc. A signal processor further communicates with the infrared detection system to receive the output signal from the detection system. The signal processor determines a measured thermal response of the portion of the subject to the thermal stimulation and compares the measured thermal response to determine a presence of an abnormality by detecting a deviation of the measured thermal response from an expected thermal response that is free of the abnormality. The signal processor can be a computer executing a computer program. An example use of the embodiment of the invention is to image cutaneous pigmented lesions etc.

The infrared detection system receives radiation emitted not only from the object but also from the surroundings, the atmosphere and the optics of the device (Hamrelius, T., 1991, Proc. SPIE, 1467, pp. 448-57). Furthermore, the intensity of the object radiation is a function of the surface emissivity of the investigated object unless the object is a perfect blackbody. The relation between the device output and the object radiance is calculated from

L=λ·ε·L⁰(Tₒ)+λ·(1−ε)·L⁰(Tₛᵤᵣ)+(1−λ)·L⁰(Tₐₜₘ), (1)

where L⁰(T) is the spectral radiance of a blackbody at temperature T, ε is the emissivity of the object, λ is the transmittivity of the atmosphere over the sensitivity range of the device and Tₒ, Tₐₜₘ, Tₛᵤᵣ are object, ambient and surrounding temperature, respectively. The aforementioned expression holds under several assumptions, however, the exact relation can still be found by experimental blackbody calibration.

The measured object radiation may be first transformed into temperature. Since skin temperature is affected by environment temperature, it is important to maintain a constant ambient temperature. Imaging distance should also be kept constant since the pixel resolutions are affected by this distance. A short distance between the object and the camera, the effect of transmittivity of the atmosphere in Eq. 1 is negligible. Therefore, the calibration is done with a blackbody at a fixed short distance from the camera and constant ambient temperature.

An example infra-red detection system being used is the Merlin midwave (3-5 μm) infrared camera (MWIR) that has a thermal sensitivity is 0.025° C. and includes a 320×256 InSb focal plane array (FPA) capable of recording with a frame rate of 60 Hz. The calibration procedure includes positioning the blackbody that is brought to different temperatures (5-35° C. with 0.5 degree increment) in front of the camera. As the temperature of the blackbody is varied stepwise, the infrared images are successively captured. The image of a distant object has the shape of a disk surrounded by concentric rings of weaker intensity, the average intensity of the central pixels (60×60) can be used to compute the calibration curve through the following polynomial fit.

T(° C.)=−53.771+0.0045575·g−1.1612·10⁻⁷·g²+1.692.10⁻¹²·g³·9.9176·10⁻¹⁸·g⁴, (2)

where g is the pixel intensity.

In terms of repeatability of the data, the temperature difference between initial and repeated data may be calculated first according to:

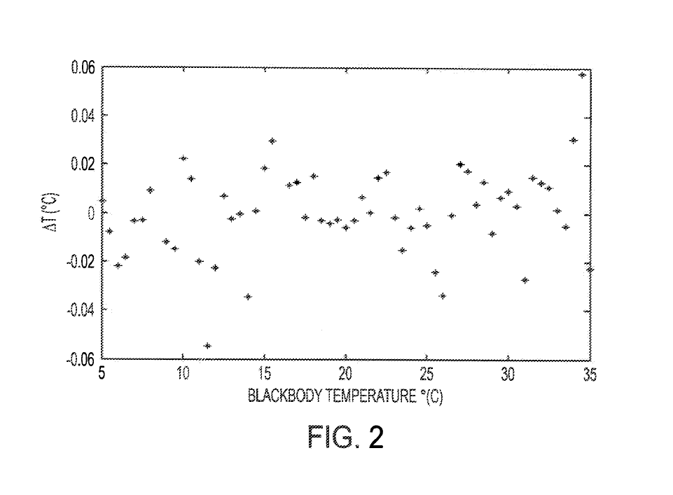

ΔT(i,j,k)=Tᵢ(i,j,k)−Tᵣ(i,j,k), (3)

where (i,j) denotes the pixel coordinates and k is the corresponding temperature value. The mean, Δ̅T̅ and standard deviation σ may be used to show the repeatability

$$\overline{Δ T} = \frac{1}{n} {\sum }_{m = 1}^{n = k} Δ {T}_{m} , \\ σ = \sqrt{\frac{1}{n} {\sum }_{m = 1}^{n = k} {[ Δ {T}_{m} - \overline{Δ T} ]}^{2}} , \qquad ( 4 )$$

where n is the number of calibration temperatures. FIG. 2 displays the temperature difference and the repeatability is calculated as 0.0023±0.02° C. (Eq. 4).

The way in which an image is formed on the detector has a direct influence on temperature measurements and it should be well understood before performing diagnosis. The point-spread function (PSF), which is a combination of aberrations, diffractions and the detector size, causes image deterioration (Maldaque, X. P., 1994, Infrared methodology and technology, Nondestructive testing monographs and tracts, 7, Gordon and Breach Science Publishers). One of the causes of deterioration is geometrical aberration. The image of a point object is a finite-sized spot, more or less widely spread around the location of the point image, which can be explained according to the laws of refraction. Since the refractive index of the camera lenses is wavelength-dependent, the camera is sensitive to a spectral range, which implies chromatic aberrations. Another cause is the diffraction which renders the image of a distant point object the appearance as a disk surrounded by concentric rings of weaker intensities. The radius of the central disk (Airy's disk) is R=1.22·τ·f/d, where τ is the wavelength, f is the focal length of the lens and d is the diameter of the lens aperture. The final cause of image deterioration is the size of the detector, which results irradiance repartition through a window of size equal to the dimension of the detector.

In order to compensate for these influences, the blackbody calibration images may be used. Since the blackbody's temperature uniformity is 0.025° C. (CI Systems SR800), it may be used to correct the image deterioration. Then the pixel based temperature error may be calculated for each calibration temperature, e(i,j,k), which is the difference between measured temperature, Tₘₑₐ, and the blackbody temperature, T_{bb} (Eq. 5). Tₘₑₐ is calculated using the calibration curve fit (Eq. 2)

e(i,j,k)=T_{bb}(k)−Tₘₑₐ(i,j,k). (5)

Since e may depend not only on the pixel position but also on blackbody temperature, multiple regression least squares method may be used first to fit a polynomial model in terms of pixel position to the temperature error based on the following Eq. 6. The method of least squares assumes that the best-fit curve of a given type is the curve that has the minimal sum of the deviations squared (least square error) from a given set of data. For a case of multiple regression least squares method where there are more than one parameter that affects the model, the data points are {i₁,j₁,e₁}, {i₂,j₂,e₂}, . . . , {iₙ,jₙ,eₙ}, where (i,j) is the independent variable and e is the dependent variable. The fitting surface f(i,j) has the deviation (error) d from each data point, i.e., d₁=e₁−f(i₁,j₁), d₂=e₂−f(i₂,j₂), . . . , dₙ=eₙ−f(iₙjₙ). Therefore, the best fitting surface has the following property:

$$min ( {\sum }_{m = 1}^{n} {d}_{m}^{2} ) = min ( {\sum }_{m = 1}^{n} {[ {e}_{m} - f ( {i}_{m} , {j}_{m} ) ]}^{2} ) . \qquad ( 6 )$$

The order of the multiple regression least-squares fitting is chosen to be 2 which uses f=c₁i²+c₂ij+c₃i+c₄j²+c₅j+c₆, to approximate the given set of data, {i₁,j₁e₁}, {i₂,j₂,e₂}, . . . , {iₙ,jₙ,eₙ} with coefficients of c₁, c₂, c₃, c₄, c₅ and c₆.

Next, since the model coefficients are changing with object temperature, a least square third degree polynomial method may be used to fit a polynomial curve to these coefficients. The least-squares third degree polynomials method uses g=z₁+z₂T+z₃T²+z₄T³ such as to approximate the given set of data, {T₁,c₁₍₁₎}, {T₂,c₁₍₂₎}, . . . , {Tₙ,c₁₍ₙ₎}.

Finally, in order to correct the images, the following steps may be included:

i) Calculate g(k) that depends on object temperature, Tₒ(k), i.e, the temperature at the center of the image

g(k)=z₁+z₂·Tₒ(k)+z₃·Tₒ(k)²+z₄·Tₒ(k)³, (7)

where z₁, z₂, z₃ and z₄ are coefficients calculated from least square 3^{rd} degree polynomials method.
ii) Calculate e using g(k) and multiple regression least-squares method

e(i,j,k)=g₁(k)·i+g₂(k)·i·j+g₃(k)·i+g₄(k)·j²+g₅(k)·j+g₆(k), (8)

where gᵢ(k), . . . , g₆(k) are coefficients calculated from Eq. 7.
iii) Add e and measured temperature, Tₘₑₐ, to find the corrected-image, T_{corr},

T_{corr}(i,j,k)=Tₘₑₐ(i,j,k)+e(i,j,k). (9)

After finding the corrected temperature fields, the mean and the standard deviation may be used to show the error, l(i,j,k), between the corrected temperature fields, T_{corr}, and the blackbody temperature according to the following Eq. 10.

$$l ( i , j , k ) = {T}_{bb} ( k ) = {T}_{corr} ( i , j , k ) \\ \overline{l} = \frac{1}{n} {\sum }_{m = 1}^{n} {l}_{m} , \\ σ = \sqrt{\frac{1}{n} {\sum }_{m = 1}^{n} {[ {l}_{m} - \overline{l} ]}^{2}} \qquad ( 10 )$$

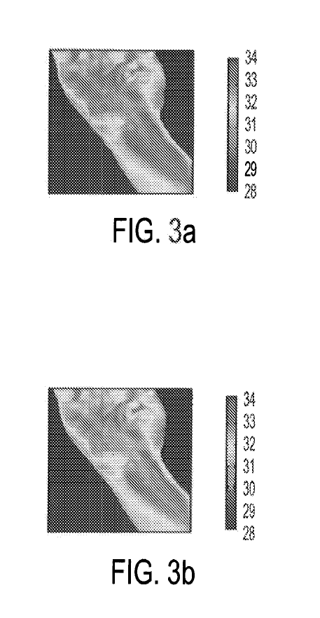

FIGS. 3a and 3b show an uncorrected temperature map and the corresponding corrected temperature map of human skin, respectively. The corrected image is obtained based on the above procedure.

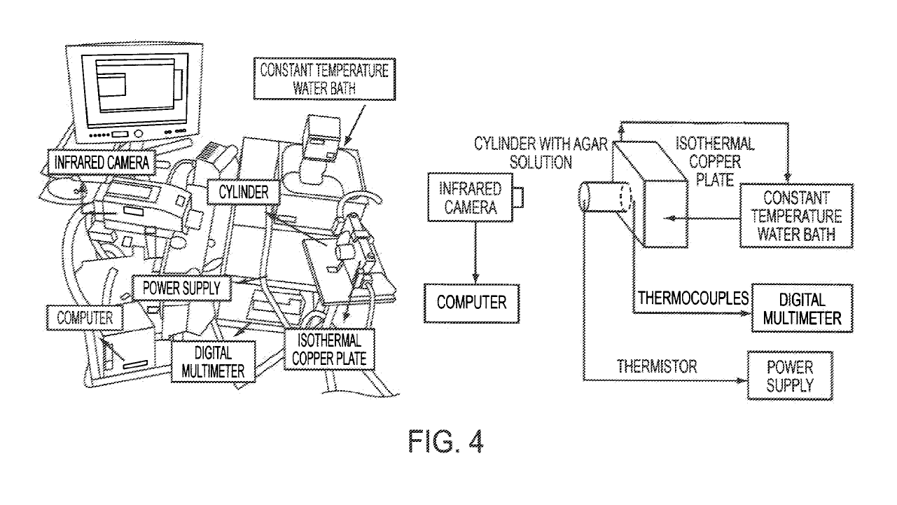

FIG. 4 shows a photograph of an experimental set-up and a schematic of the set-up. The phantom in the experimental set-up comprises a garolite hollow cylinder filled with the agar solution-mounted on a rectangular copper plate serving as the constant temperature surface that remains at the core body temperature. The copper plate may be equipped with several channels through which water can be pumped from a constant temperature water bath. In this way, the temperature of the plate and the base of the cylinder filled with the agar remain at 37° C., the core body temperature. The thermocouples are utilized to monitor the temperature of the copper block and the interface between the copper block and the agar as well as the surface of the agar. The uniformity of the copper block temperature is verified using the infrared camera and temperature measurements. The average variation of copper plate temperature in the region of the cylinder is found to be 0.05° C. The skin phantom is prepared by slowly dissolving the 4.0% solution of DIFCO AGAR TECHNICAL in boiling water. The agar solution is allowed to cool for a few hours until it has jelly-like appearance, and then poured into the cylinder. The outer diameter, the wall thickness and the height of the cylinder are 50 mm, 1.5 mm and 25 mm, respectively, as shown in FIG. 4. After the cylinder is filled with the agar solution, the thermistor, which represents a lesion, is immersed into the solution.

The thermistor is connected to a power supply that allows adjusting the voltage applied across it. As reported by Draper and Boag (Draper J W and Boag J W 1971, Phys. Med. Biol. 16(4) 645-56), the heat generation rate of a healthy tissue is 700 W/m³, and that of a tumor is no more than 25,000 W/m³. Different heat dissipation values are achieved in our experiment by varying the power supplied to the thermistor. Since the resistance of the thermistor changes with the surrounding temperature, during the cooling phase heat dissipation or the temperature profile may not constant. However, in the numerical model, the temperature boundary condition along the lesion perimeter is defined as constant. Nevertheless, the temperature distribution is expected to be consistent with patterns in the numerical model.

Using the same principle as in the numerical model, the time evolution of the infrared signal may be analyzed after a cooling or heating stress is applied to the skin phantom model. Cooling stress is applied by blowing cold air using an Exair vortex tube inside the cylindrical apparel attached on the agar surface (FIG. 4). After removing it, the transient thermal response of the surface is captured.

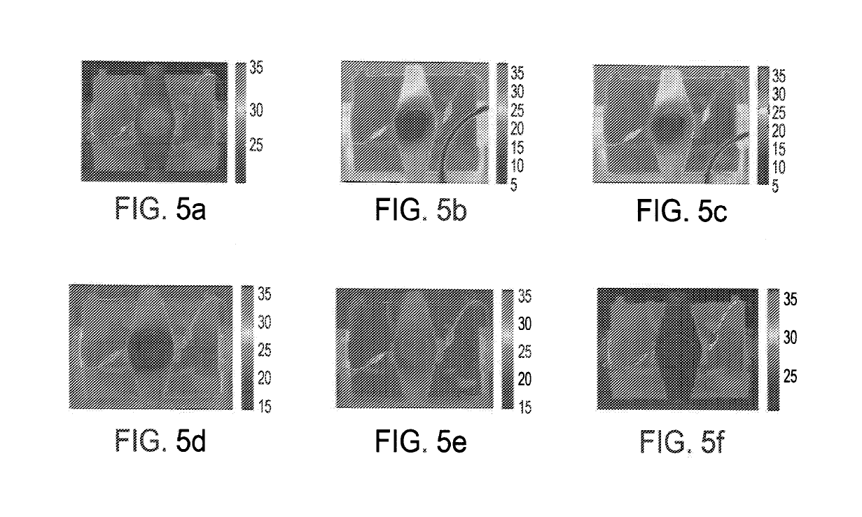

FIGS. 5a-5f display the temperature fields of the infrared images captured from the skin phantom shown in FIG. 4. FIG. 5a shows the temperature map of the phantom at steady state. FIG. 5b shows the temperature map of the phantom having undergone a cooling excitation of 120 s. FIG. 5c-5f show the temperature maps of the phantom during recovery at 30 s, 90 s, 400 s and 720 s after the cooling excitation, respectively.

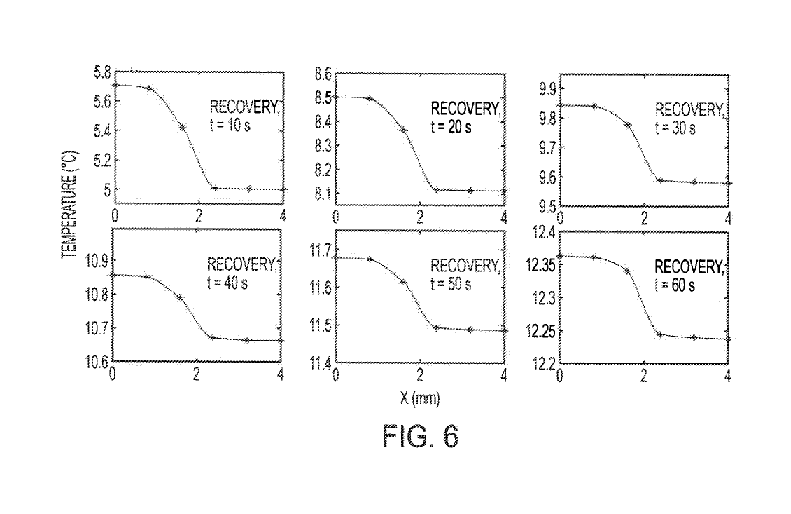

Since the phantom model is symmetric, the temperature profiles of the agar surface are defined along the line from the origin (0,0) to (8-mm,0). FIG. 6 shows these profiles for selected recovery times. The largest temperature changes occur within the first few minutes after the cooling is removed. By considering both the steady state and transient results, information about the size and depth of masses within the skin phantom is recovered.

Human skin can be modeled using the bioheat equation by Pennes (Pennes H H 1984, J. Appl. Physiol. 1 93-122), which is a transient heat conduction equation of the form

$$ρ C \frac{\partial T}{\partial t} = k {∇}^{2} T + {ρ}_{b} {C}_{b} {w}_{b} ( {T}_{b} - T ) + {Q}_{met} \qquad ( 11 )$$

where σ is the tissue density, C is the specific heat of the tissue, T is the local tissue temperature, k is the thermal conductivity of the tissue, σ_{b} is the blood density, C_{b} is the specific heat of the blood, T_{b} is the arterial blood temperature, w_{b} is the blood perfusion rate and Qₘₑₜ is the metabolic heat generation per unit volume. Eq. 11 states that the rate of change of thermal energy contained in a unit volume is equal to the sum of the rates at which thermal energy enters/leaves the unit volume by i) conduction, ii) perfusion, and iii) metabolic heat generation. In a simplified numerical model, the term describing the metabolic heat generation may be neglected.

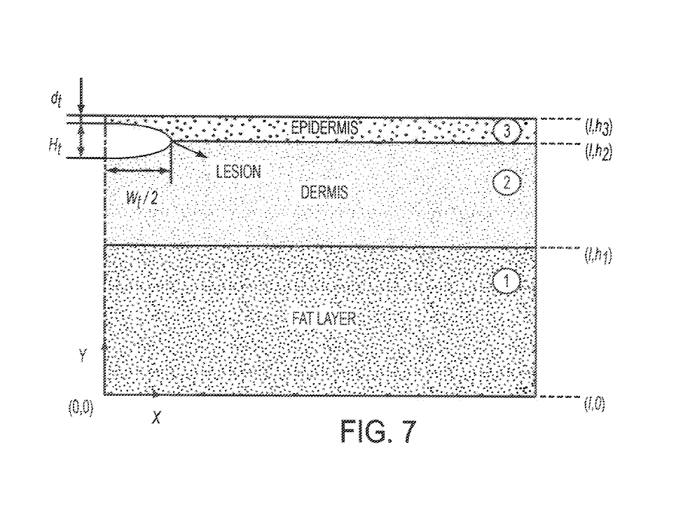

FIG. 7 shows a model of a skin lesion having three layers, namely, the epidermis, dermis, and fat layer. The model can easily be refined to have more layers, as needed. Each layer of the skin tissue is modeled as an infinitely spanning homogeneous medium of finite thickness in the y direction and infinite in the x and z direction, characterized by a set of thermophysical properties subject to sensitivity analysis in this study. The expression in Eq. 11 describes the temperature distribution in each of the three tissue layers. In each region n, the temperature is found by

$${ρ}_{n} {C}_{n} \frac{\partial {T}_{n}}{\partial t} = {k}_{n} {∇}^{2} {T}_{n} + {ρ}_{b} {C}_{b} {w}_{b} ( {T}_{b} - {T}_{n} ) + {Q}_{met}$$

for n=1 . . . 3 over the interval hₙ₋₁<y<hₙ, h₀=0. (12)

Eq. 12 can be solved by imposing boundary conditions at the surfaces and continuity conditions on the temperature and heat flux at each interface between tissue layers. Assuming no heat flux from the both side of the layers (x=l and x=0), the boundary condition takes the from

$$\frac{\partial T}{\partial x} {\vert}_{x} = 0 at x = 0 , x = l . \qquad ( 13 )$$

The interface temperature continuity condition is written as:

Tₙ(hₙ,t)=Tₙ₊₁(hₙ,t) (14)

while for the conservation of heat flux is:

$$- {k}_{n} \frac{\partial {T}_{n}}{\partial y} {\vert}_{y} = - {k}_{n + 1} \frac{\partial {T}_{n + 1}}{\partial y} {\vert}_{y} at y = {h}_{n} . \qquad ( 15 )$$

Table 1 summarizes some of the parameters reported in Torvi and Dale (Torvi D A and Dale J D 1994, J. Biomech. Eng. 116 250-55) such as specific heat C, thermal conductivity k, density ρ, and specific heat of the blood C_{b}, along with the ranges of other parameters that may be considered.

TABLE 1
Properties of skin layers.

| | Epidermis | Dermis | Fat layer |
|---|---|---|---|
| C (J/kgK) | 3578-3600 | 3200-3400 | 2288-3060 |
| k (W/mK) | 0.21-0.26 | 0.37-0.52 | 0.16-0.21 |
| ρ (kg/m³) | 1200 | 1200 | 1000 |
| C_{b} (J/kgK) | 3770 | 3770 | 3770 |
| w_{b} (1000/s) | 0 | 6-12.5 | 6-12.5 |
| h (mm) | 0.08-0.1 | 2-3 | 8-10 |

Femlab, a commercial software package by Comsol (Comsol Multiphysics 2006 Version 3.2b Comsol Inc.) may be used to solve the coupled differential equations for these three skin layers. Since the mathematical model is not very challenging computationally, a commercial code yielding good results may be used so that the focus may be placed on the physics aspects of the problem rather than writing a dedicated computer code. Other computer codes can also be used to solve the mathematical model.

Elder (Elder D 1999, Acta Onc. 38 535-47) considers lesions as generally symmetric structures. In accordance with that work, a 2D axisymmetric model may be built and lesion may be represented as an elliptical region in the cross section shown in FIG. 7. The elliptical lesion is an example, the model can easily accommodate any lesion shape. In order to create semi-infinite tissue layers, the model may be made large enough in the lateral direction to render the thermal effects of the lesion negligible at the lateral boundaries.

Skin lesions are considered to be in Stage I & II malignant lesions of less than or equal to 2 mm in thickness (Balch C M et al 2001, J. Clin. Onc. 19 3635-48). These lesions are characterized by uncontrolled growth of melanoctyes, which are located in the stratum basale of epidermis (0.02-0.1 mm below the surface). A representative model of the skin tissue with a lesion embedded into the epidermal and dermal layers is shown in FIG. 7. The shape of the skin lesions is modeled with a width Wₜ=2 mm, a height Hₜ=0.5 mm, and the ellipses are located starting in the epidermis at dₜ=0.02 mm depth below the surface.

As mentioned previously, increased metabolic activity in a cancerous lesion causes an increase of local temperature, whereas non-cancerous lesions with skin discoloration exhibit lower metabolic activity and temperature. A highly vascularized skin tumor may also cause increased local skin temperature that can be several degrees higher than that of the surrounding tissue (Draper J W and Boag J W 1971, Phys. Med. Biol. 16(4) 645-56; Deng Z and Liu J 2004, Comp. Bio. Med. 34 495-521). Thus, the lesion can be represented by, for example, an elliptical region with a constant temperature boundary condition prescribed along its perimeter (Draper J W and Boag J W 1971, Phys. Med. Biol. 16(4) 645-56). The lesion boundary is also assumed to be 0.5 degrees warmer than its surrounding in accordance of the studies by Lawson (Lawson R 1956, Can. Med. Assoc. 1 75 309-10), Draper and Boag (Draper J W and Boag J W 1971, Phys. Med. Biol. 16(4) 645-56), and Deng and Liu (Deng Z and Liu J 2004, Comp. Bio. Med. 34 495-521).

A different representation would be to describe the lesion as an elliptical region of increased metabolic activity characterized as heat source in the mathematical model. This option may also be included in our study as one of the model parameters since measurement data regarding metabolic heat generation rates may become available. The computational model as an embodiment of the invention can be easily refined using additional information on the thermal and thermophysical properties that would be available with time, as the knowledge base increases.

The thermal conductivity of a skin lesion was found to be approximately 89% that of water (Ahuja A S, Prasad K N, Hendee W R and Carson P L 1978, Med. Phys. 5(5) 418-21). Assuming a standard core body temperature of T_{c}=37° C. (Torvi D A and Dale J D 1994, J. Biomech. Eng. 116 250-55) and a specific heat and density approximately comparable to the properties of water, the following skin lesion properties were obtained: kₜ=0.558 W/m K; Cₜ=3852 J/kg K; ρₜ=1030 kg/m³.

First, it may be assumed that for time t<0, steady state conditions within the tissues are arising from the top skin surface that is exposed to convective boundary condition. Therefore, the boundary condition at the top surface is

q′=h_{∞}(T(x,y,t)−T_{∞}) at y=h₃

h_{∞}=5 W/m²K, T_{∞}=21° C. (16)

where h_{α} is heat transfer coefficient and T_{α} is ambient temperature. The bottom surface of the fat layer may be assumed to be at constant temperature boundary condition,

T(x,y,t)=T_{c} at y=0,

T_{c}=37° C. (17)

where T_{c} is core body temperature. This solution serves as the initial condition to study the effects of cooling. At time t=0, to achieve cooling, a prescribed surface temperature boundary condition,

T(x,y,t)=Tₛ at y=h₃,

Tₛ=4° C. (18)

may be applied to the top surface. The skin is cooled for duration of 120 s. At time t=120 s, the constant temperature boundary condition is removed, and the surface is again exposed to convection. These numbers are examples only and they can vary from case to case, depending on the situation. The skin is then allowed to return to its original temperature, which is called the recovery process. It takes approximately 1500 s for the skin to reach its original steady state condition.

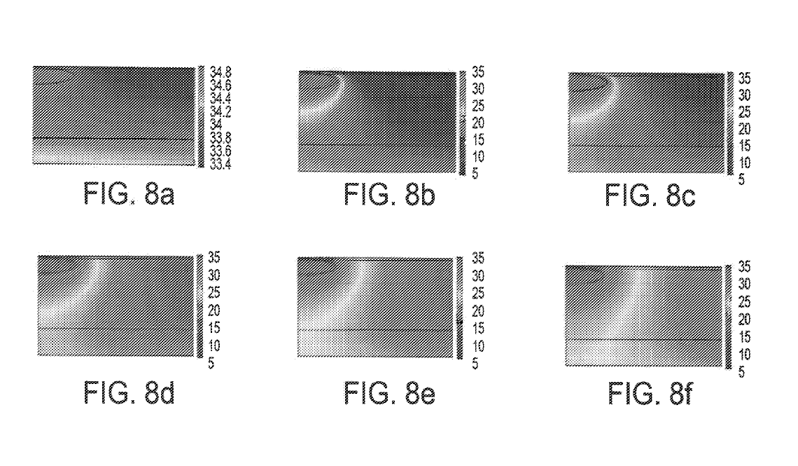

FIG. 8a shows the cross-sectional temperature map of the model in FIG. 7 during steady state. FIG. 8b shows the cross-sectional temperature map of the model in FIG. 7 after a 120 s of cooling excitation. FIGS. 8c-8f show the cross-sectional temperature maps of the model in FIG. 7 during recovery at 15 s, 30 s, 45 s, and 60 s after the cooling excitation. When the skin layers are subjected to cooling, the change in the temperature of the skin can be observed at different depths. After the removal of the cooling stress, it is observed that the largest changes in temperature occur within the first few minutes. Therefore, the temperature distribution is displayed at different recovery times particularly at earlier times. It takes approximately 1500 s for the skin to reach its steady state.

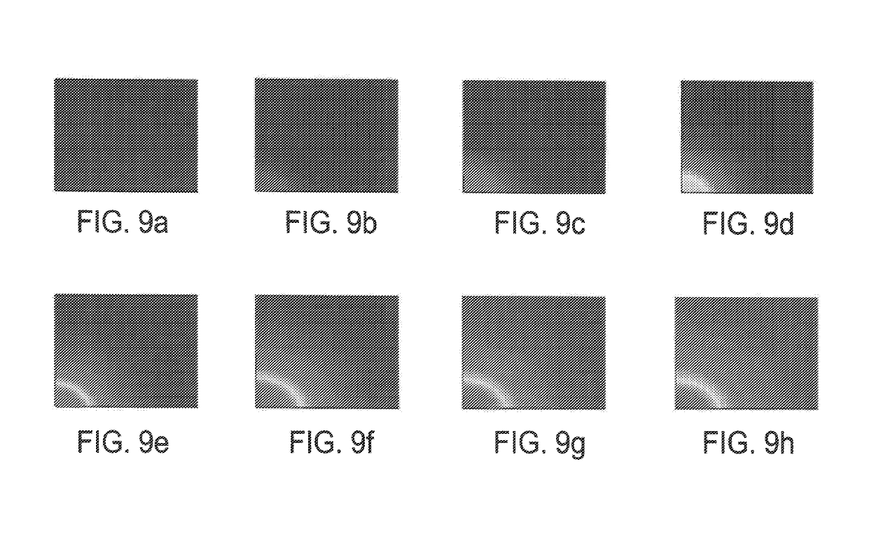

FIGS. 9a-9h show the surface temperature maps of the model in FIG. 7 during recovery at 15 s, 30 s, 45 s, and 60 s after the cooling excitation. The images illustrate the speed at which natural convection heats the skin. Thus, the largest changes in temperature are observed within the first minute minutes after the removal of the cooling stress.

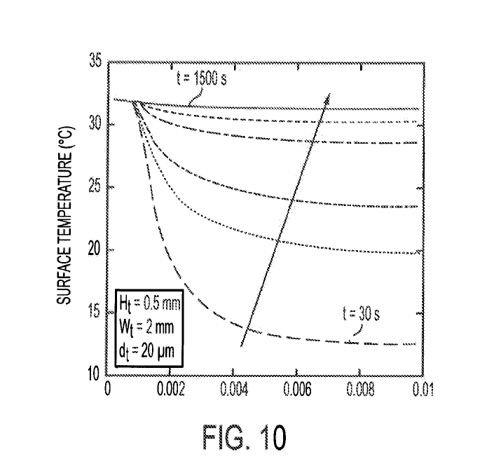

FIG. 10 shows surface temperature profiles of a 2 mm width (Wₜ), 0.5 mm height (Hₜ) and 20 μm depth (dₜ) lesion. Each line represents a particular recovery time.

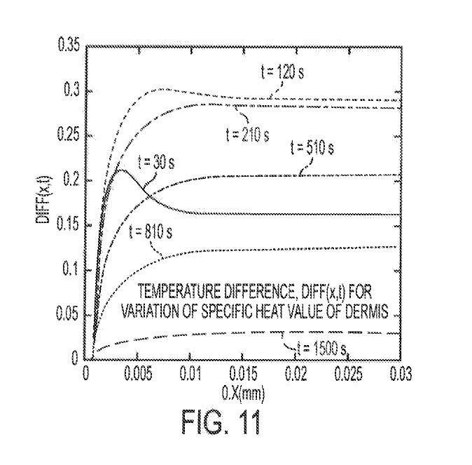

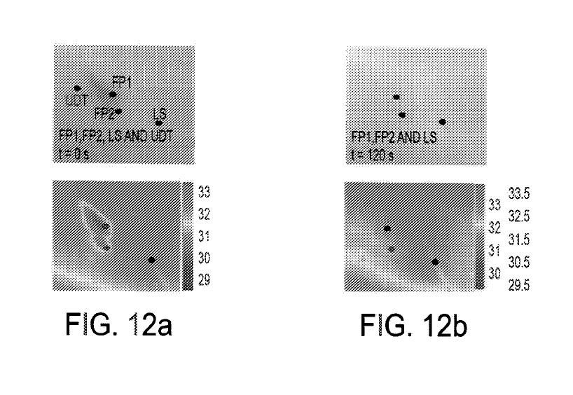

FIG. 11 shows the temperature difference, diff(x,t), for varying values of the specific heat of the dermis at different recovery times. Recalling that the largest changes in temperature occur within the few minutes after the removal of the cooling stress, the difference between the resulting surface temperature distributions is largest within the first few minutes (˜2 minutes). As the skin reaches its steady state, the temperature difference decreases.

The effects of varying the values of specific heat, thermal conductivity, blood perfusion rate and thicknesses of the skin layers on surface temperature are investigated as a part of the sensitivity study. At each experiment, the value of the selected parameter of interest is varied within its range given by table 1, while those of other parameters are kept constant at their default values given by table 2. Default values of the specific heat and thermal conductivity are taken to be the mid-values of their respective ranges.

TABLE 2
Default properties of skin layers

| | Epidermis | Dermis | Fat Layer |
|---|---|---|---|
| C (J/kgK) | 3589 | 3300 | 2674 |
| k (W/mK) | 0.235 | 0.445 | 0.185 |
| ρ (kg/m³) | 1200 | 1200 | 1000 |
| C_{b} (J/kgK) | 3770 | 3770 | 3770 |
| w_{b} (1000/s) | 0 | 12.5 | 12.5 |
| h (mm) | 0.08 | 2 | 10 |

Each parameter is tested at its extreme values for each layer, while keeping the other parameters constant at their default values. During recovery phase, the resulting surface temperature distributions for each parameter's extreme values are obtained. In this way for each parameter, time series, i.e. a sequence of data points measured typically at successive time instances, may be generated. To analyze these time series, Eq. 19 can be used as follows: first, the difference between the resulting surface temperature distributions, diff(x,t), is calculated; then, the standard deviation of this difference, std(x), is computed with respect to time; finally, the maximum standard deviation, max(std(x)), may be used as a measure of parameter sensitivity.

$$\begin{matrix}diff ( x , t ) = {T}_{i} ( x , t ) - {T}_{s} ( x , t ) \\ std ( x ) = std ( diff ( x , t ) ) = \sqrt{\frac{1}{N} ( {\sum }_{i = 1}^{n} {( {diff}_{i} ( x , t ) - {diff}^{-} ( x , t ) )}^{2}} & ( 19 ) \\ max ( std ( x ) ) = maximum of \{ std ( x ) \} & \end{matrix}$$

The results of the sensitivity study on the specific heat and the thermal conductivity at different tissue layers are summarized in table 3. As the variations in these thermophysical properties are relatively small for the individual layers, variations in temperature are found to be very small as well.

TABLE 3
Standard deviation of temperature differences for variation
thermophysical properties

| | C (J/kgK) | max(std(x)) | k (W/mK) | max(std(x)) |
|---|---|---|---|---|
| Epidermis | 3578-3600 | 0.003 | 0.21-0.26 | 0.065 |
| Dermis | 3200-3400 | 0.09 | 0.37-0.52 | 0.25 |
| Fat Layer | 2288-3060 | 0.2 | 0.16-0.21 | 0.12 |

The results at varying values of the blood perfusion rate and the thicknesses are outlined in table 4, which demonstrates that the perfusion rate and the thicknesses have little effect on surface temperature distribution.

TABLE 4
Mean temperature differences for the blood perfusion
rate and thickness variation

| | w_{b} (1000/s) | max(std(x)) | h (mm) | max(std(x)) |
|---|---|---|---|---|
| Epidermis | 0 | 0 | 0.08-0.1 | 0.035 |
| Dermis | 6-12.5 | 0.2 | 2-4 | 0.19 |
| Fat Layer | 6-12.5 | 0.22 | 8-10 | 0.1 |

During numerical analysis, a reheating index, describing the recovery to equilibrium, after the initial thermal stimulation, may be used. The reheating index may be derived empirically or via parametric model fitting.

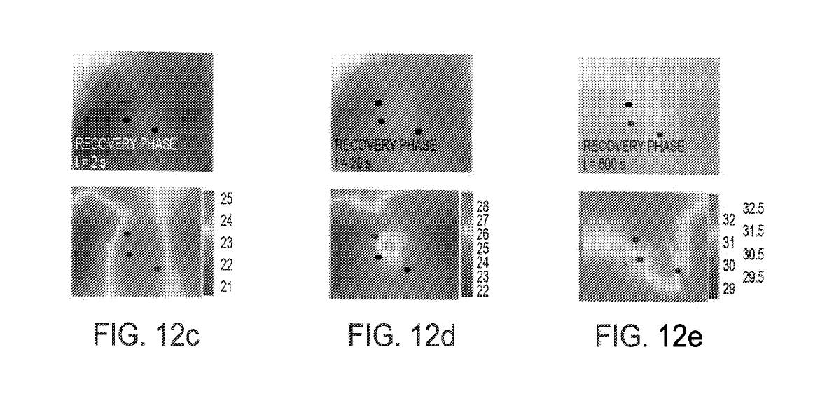

Infrared imaging experiments were also conducted in a laboratory setting on healthy human skin tissue using an embodiment of the current invention. Sample infrared thermographic images, for example, can be subjected to a number of filtering operations to enhance the desired features and covert the grayscale information into color-coded temperature are shown in FIG. 12. During the experiments, focal pressure was applied to healthy tissue at two locations, shown as focal points FP1 and FP2 in FIG. 12. A line scratch LS was also applied to healthy tissue as shown in FIG. 12. FIG. 12a shows focal points FP1, FP2, and line scratch LS as clearly visible in the infrared image at the start of applying a thermally cooling stimulation. FIG. 12b shows FP1, FP2 and LS nearly disappearing 120 s after application. At 120 s the cooling stress is applied for 2 minutes. FIGS. 12c-e show the skin during the thermal recovery phase after 2 s, 20 s and 600 s. From these images, the cooling stress obviously enhances the contrast between the features of FP1, FP2, and LS and those of the undisturbed healthy tissue. FP1, FP2 and LS again become visible in the infrared image. These three disturbances simulate the increased temperature of the cancerous skin lesion. A similar approach was used to successfully identify basal cell carcinoma (Buzug, T. M., Schumann, S., Pfaffmann, L., Reinhold, U. and Ruhlmann, J., 2006, IEEE EMBS, 2766-2769).

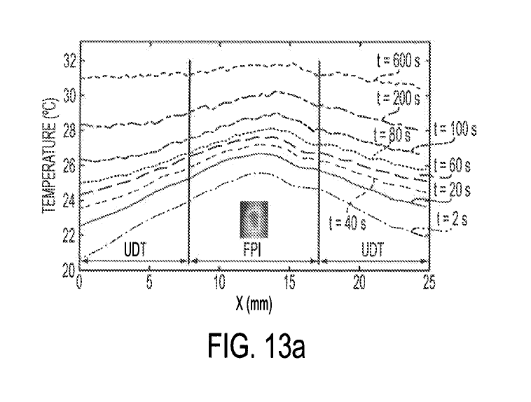

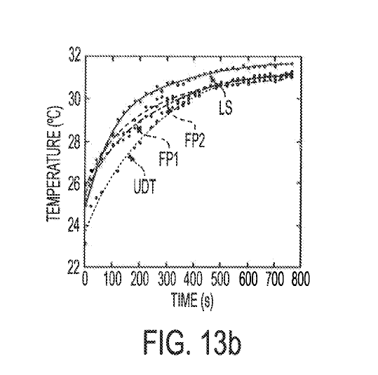

FIG. 13a shows temperature profiles measured at different time instants during thermal recovery (each curve corresponds to a time instant from 2 s to 600 s) in a skin cross section encompassing regions of undisturbed tissue UDT and the region of the focal point FP1. The temperature difference between FP1 and UDT is very pronounced shortly after the removal of the cooling stress and decreases with time. FIG. 13b shows temporal temperature distributions for FP1, FP2, LS and undisturbed tissue UDT. There is a distinct difference between the temporal temperature distribution of undisturbed tissue UDT and those of FP1, FP3 and LS and this difference can be used to determine a reheating index to differentiate, for example, an underlying abnormality from normal physiology. The results shown in FIGS. 13a and 13b thus demonstrate the mechanism of some embodiments of the present invention.

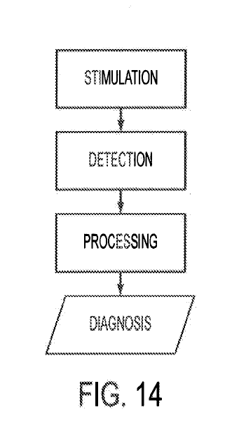

FIG. 14 shows a flow chart of another embodiment of the invention as a method diagnosing a suspected abnormality. The method comprises: thermally stimulating at least a portion of a subject under observation having said suspected abnormality; detecting infrared radiation to provide an output signal from said at least a portion of said subject after said thermally stimulating; processing said output signal to compare a measured thermal response of said portion of said subject after said thermally stimulating to an expected thermal response to determine a presence of said abnormality. Some embodiments of the present invention may be used both for the local imaging of a lesion and total body imaging, nowadays usually accomplished by digital photography.

The thermally stimulating may comprise a cooling or heating excitation and may be modulated. The measured thermal response can be analyzed numerically to quantify a parameter, which may be at least one of: a size, a depth, a quantity indicative of a metabolic activity, or a reheating index. The reheating index, describing the recovery to equilibrium after the thermal stimulation, may derived empirically or via parametric model fitting. The numerical analysis may be according to a layered bioheat equation similar to Eq. 12.

Some embodiments of the invention may allow for the rapid, quantitative assessment of thermal changes in the skin over time. Such assessment of thermal changes is expected to be significantly altered in cutaneous disorders associated with primary or secondary heat generation either through direct proliferative effects in the skin or subcutaneous tissues, or indirect heat generation via changes in vascular perfusion of cutaneous/subcutaneous regions of the skin and/or inflammation within the cutaneous/subcutaneous regions of the skin. The rapid quantification of thermal changes in the skin may be of tremendous utility in the diagnosis of various cutaneous disorders, prediction of treatment responses for various primary or secondary skin diseases, and prediction of clinical outcomes of primary or secondary cutaneous disorders. The use of such a tool with objective, quantifiable parameters will allow for standardization of diagnostic/prognostic/therapeutic algorithms both for a particular individual and also for large numbers of individuals with particular cutaneous disorders. Such a diagnostic/prognostic tool is expected to improve cost-effective treatment of cutaneous disorders and allow for rapid, early diagnosis of cutaneous disorders including skin cancers which will allow for significant decreases in associated morbidity and mortality.

Specific examples of the utility of the disclosed embodiments of the invention in primary and secondary cutaneous disorders may include the following conditions: pigmented lesions and melanoma, non-melanoma skin cancers, vascular disorders of the skin, primary inflammatory/autoimmune diseases of the skin, secondary inflammatory diseases of the skin, primary/secondary infectious disease, disorders of aging, etc.

Melanomas are notorious for their ability to metastasize at a relatively early stage of development and the key to improved survival in all affected individuals remains early diagnosis and treatment. The vast majority of cutaneous melanomas present as pigmented lesions in the skin, and current detection of atypical lesions relies primarily on subjective criteria including the A (Asymmetry), B (Borders), C (Color), D (Diameter), E's (Evolution) of melanoma. Malignant pigmented lesions with increased proliferative potential generate quantifiable amounts of heat and possess an ability to reheat more quickly than surrounding normal skin. Some embodiments of the invention allow for precise measurement of warming variations in the skin which may be used to evaluate cutaneous pigmented lesions using a quantifiable, objective unit of measure. Some embodiments may be further optimized to allow for detection of high-risk pigmented lesions with a significant malignant potential versus low-risk lesions of minimal malignant potential. These quantitative determinations will allow for accurate identification of lesions requiring excision and histopathologic evaluation. Some embodiments of the invention may significantly enhance screening of persons with a significant risk for developing melanoma including those with an increased number of nevi (moles), those with irregular (dysplastic) nevi, those with a personal history of a previous history of melanoma, those with a family history of melanoma, and individuals of fair complexion with decreased tanning ability as well as individuals with a history of previous sunburns during childhood and adolescence. Some embodiments of the invention may allow for significant reductions in the number of skin biopsies being performed for benign pigmented lesions and subsequent reductions in associated morbidities and healthcare costs. Moreover, some embodiments of the invention will allow for earlier detection of skin cancers at their most curable point reducing the mortality associated with more invasive, later stage melanomas. Particular utility is anticipated in the tracking of large pigmented lesions like Giant Congenital Nevi in children which cover a significant percentage of the skin surface, are intractable to complete surgical removal, and possess a significant risk for malignant conversion. Current protocols use bright light images to track surface changes in these lesions which may be subtle in the face of malignant conversion. An objective measure of thermal profiles of such lesions with serial imaging will allow for early detection of metabolic changes associated with biologic alterations including conversion to a more aggressive and/or malignant state.

Non-melanoma skin cancers can also benefit from some embodiments of the invention for more accurate detection of early malignant lesions and improved delineation of tumors margins for surgical considerations. Non-melanoma skin cancers may include Basal Cell Carcinoma (BCC), Squamous Cell Carcinoma (SCC), Cutaneous Lymphomas, Merkel Cell Carcinomas, Histiocytosis, Leukemia Cutus, other primary or secondary cutaneous malignancies, hamartomatous lesions with cancer risk (e.g. Nevus Sebaceous), etc. Additional utility may be gained from the quantitative analysis of individual lesions which may serve as predictors of disease outcome and/or response to therapy. In the case of benign lesions (hamartomatous nevi) with a significant risk for malignant conversion, serial thermal images will allow for early detection of premalignant/malignant changes through identification of altered thermal profiles.

A large number of cutaneous vascular lesions are seen in both children and adults. Congenital vascular lesions including hemangiomas, port-wine stain lesions, and other vascular abnormalities may have variable courses over time and variable responses to therapy. It is anticipated that lesions with a propensity to involute sponteously will possess an altered thermal profile versus a lesion with a propensity to grow over time and that lesions with significant proliferative potential will generate increased thermal energy. Such lesions may benefit from serial thermal imaging using embodiments of the invention to guide therapeutic decision-making including timing and nature of therapies to be used in a particular case. Additional vascular lesions which remain stable over time may also benefit from single or serial thermal imaging to predict outcome and/or response to therapies. Such therapies may include laser therapies, intralesional steroid therapies, oral systemic agents where thermal imaging may be predictive or particular therapeutic response to a single agent over others, or the non-responsiveness of a lesion to any therapeutic option with the exception of surgical intervention. Such an imaging device will allow for decreased morbidity associated with therapies of minimal benefit and significant toxicities, and optimal timing of therapies to decrease overall disease-associated morbidity.

Primary inflammatory/autoimmune diseases of the skin may include psoriasis, eczematous dermatitis, seborrheic dermatitis, lichenoid dermatitis, pityriasis, pyoderma gangrenosum, bullous pemphigoid, pemphigus vulgaris, other autoimmune disorders of skin. Numerous inflammatory diseases of the skin are associated with significant erythema and heat generation at the skin surface. It is anticipated that such thermal changes are a reflection of the extent and severity of the primary disease. It is further suggested that thermal profiles of particular lesions may be predictive of disease course and/or disease response to therapy. As many therapeutic options exist for primary inflammatory skin diseases, patients would significantly benefit from a predictive thermal image using embodiments of the invention that would allow for identification and implementation of the most effective therapies for a particular disease with predicted therapeutic response. As many topical and systemic therapies for primary inflammatory skin diseases possess significant morbidities, thermal imaging using embodiments of the invention will allow for decreased morbidity associated with therapies of minimal benefit and optimal timing of therapies to decrease overall disease-associated morbidity. As the primary cutaneous bullous disorders may be associated with significant morbidity and mortality, patients with these particular disorders would benefit from rapid prediction of disease treatment response and initiation of optimal therapies in an expedited fashion.

Secondary inflammatory diseases of the skin may include autoimmune lupus, scleroderma, dermatomyositis, Steven's-Johnson syndrome, erythema multiforme, toxic epidermal necrolysis, staph scalded skin syndrome, pyoderma gangrenosum, urticaria, vasculitis, drug hypersensitivity reactions, etc. Systemic inflammatory disorders often possess specific cutaneous manifestations which are readily detectable and may be a significant component of the overall disease process. As thermal imaging using embodiments of the invention for cutaneous lesions over time may be predictive of disease outcome both in the skin and in other organs, the imaging results will allow for the rapid prediction of disease response to particular therapies and therefore the rapid implementation of the most effective therapies. As these disorders may be associated with significant morbidity and mortality, patients with these particular disorders would benefit from rapid prediction of disease treatment response and initiation of optimal therapies in an expedited fashion.

Primary/secondary infectious disease may include human papillomavirus (HPV)/warts, herpes simplex, varicella zoster, molluscum contagiosum, folliculitis, acne vulgaris, additional bacterial/viral/fungal infections, etc. Infectious lesions in the skin may be quite burdensome and can be associated with significant morbidity and occasional mortality. Common cutaneous lesions associated with infectious agents include acne vulgaris, HPV-associated infection (warts), mollluscum contagiosum, folliculitis, and other bacterial/viral/fungal infections. As these disorders have variable courses, with some remitting spontaneously and others progressing to widespread fulminant disease, an imaging modality using embodiments of the invention that can predict disease course and/or treatment response can be highly beneficial in improving disease treatment and decreasing disease-associated morbidities. As the immune response and associated heat generation incurred with inflammation may be evaluated and quantified using our high-resolution thermal imaging device, such images will allow for prediction of disease course over time and therapeutic responses. As an example, children may develop several hundred lesions of molluscum contagiosum. Although unsightly, these lesions are rarely problematic and most often remit over time. As the most effective treatment for these lesions is quite painful and rarely tolerated by young children, an imaging technology that could predict the nature and timing of lesional course would be extremely beneficial to patients avoiding unnecessarily painful therapies. It would also provide significant relief to anxious patients. In the case of acne vulgaris, there are cases that remain mild and relatively self-limited, which other cases go on the more widespread disease with associated scarring. The ability to identify patients that have a significant risk for developing aggressive disease and would benefit from the most aggressive treatments early on would allow for improved cosmetic results and decreased morbidity associated with failed therapeutic interventions in addition to decreased anxiety associated with the stigma of this particular disease. In other instances, patients may never completely clear their acne following their teenage years and will develop “chronic, adult” acne. The ability to predict such an outcome with thermal imaging would be of significant benefit to patients in order to develop a long term management plan with informed patient expectations.

Over time, the skin tends to lose its elasticity and turgor. Such changes in skin thickness and strength are commonly associated with changes is heat retention and are expected to result in altered infrared imaging over time. As thermal imaging using embodiments of the invention may be used to grade the amount of “skin aging” changes including skin thinning and subtle skin textural changes, serial thermal imaging using embodiments of the invention may be used to identify aging changes in the skin and the pace of such changes over time. Such images may be used to guide treatment of age-related skin changes including the use of topical therapies to improve skin tone and turgor or more invasive procedures including laser treatments such as laser resurfacing or dermabrasion techniques, chemical peeling, or additional surgical interventions. As thermal imaging using embodiments of the invention will allow for the detection of subtle epidermal and dermal changes resulting in altered heating and cooling properties of the skin, such images may be used to identify the most suitable treatment options for aging skin that will result in the best cosmetic results with the least morbidity.

In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A medical diagnosis system, comprising:
a thermal stimulator;
an infrared detection system constructed and arranged to detect infrared radiation from at least a portion of a subject under observation to provide an output signal from said portion of said subject after undergoing thermal stimulation from said thermal stimulator; and
a signal processor in communication with said infrared detection system to receive said output signal from said infrared detection system,
wherein said signal processor determines a measured thermal response of said portion of said subject to said thermal stimulation and compares said measured thermal response to an expected thermal response to determine a presence of an abnormality.

2. The medical diagnosis system of claim 1, wherein said abnormality is directed to at least one of a proliferative disease, a cutaneous vascular lesion, an inflammatory disease, an autoimmune disease, an infectious disease, or an aging skin.

3. The medical diagnosis system of claim 1, wherein said thermal stimulator delivers a cooling excitation.

4. The medical diagnosis system of claim 1, wherein said thermal stimulator delivers a heating excitation.

5. The medical diagnosis system of claim 1, wherein said infrared detection system comprises an imaging detector.

6. The medical diagnosis system of claim 1, wherein said processor is a computer executing a computer program.

7. The medical diagnosis system of claim 1, wherein said measured thermal response is analyzed numerically to quantify a parameter.

8. The medical diagnosis system of claim 7, wherein said parameter is at least one of a size, a depth, a quantity indicative of a metabolic activity, or a reheating index.

9. The medical diagnosis system of claim 8, wherein said reheating index is derived empirically.

10. The medical diagnosis system of claim 8, wherein said reheating index is obtained by parametric model fitting.

11. The medical diagnosis system of claim 7, wherein said measured thermal response is analyzed numerically according to a layered bio-heat equation based on the following formula or variations thereof:
$${ρ}_{n} {C}_{n} \frac{\partial {T}_{n}}{\partial t} = {k}_{n} {∇}^{2} {T}_{n} + {ρ}_{b} {C}_{b} {w}_{b} ( {T}_{b} - {T}_{n} ) + {Q}_{met} ,$$
wherein n is the n^{th} layer, ρc is thermal capacity of tissue, T is spatial temperature distribution in tissue, k is thermal conductivity of tissue, ρ_{b}c_{b} is thermal capacity of blood, w_{b} is blood perfusion rate, T_{b} is spatial temperature distribution in blood, and Qₘₑₜ, is the metabolic heat generation per unit volume.

12. A method of diagnosing, comprising:
thermally stimulating at least a portion of a subject under observation having a suspected abnormality;
detecting infrared radiation to provide an output signal from said at least a portion of said subject after said thermally stimulating;
processing said output signal to compare a measured thermal response of said portion of said subject after said thermally stimulating to an expected thermal response to determine a presence of said abnormality.

13. The method of claim 12, wherein said suspected abnormality is associated with at least one of a proliferative disease, a cutaneous vascular lesion, an inflammatory disease, an autoimmune disease, an infectious disease, or an aging skin.

14. The method of claim 12, wherein said thermally stimulating comprises a cooling excitation.

15. The method of claim 12, wherein said thermally stimulating comprises a heating excitation.

16. The method of claim 12, wherein said thermally stimulating is modulated.

17. The method of claim 12, wherein said measured thermal response is analyzed numerically to quantify a parameter.

18. The method claim 17, wherein said parameter is at least one of a size, a depth, a quantity indicative of a metabolic activity, or a reheating index.

19. The method of claim 18, wherein said reheating index is derived empirically.

20. The method of claim 18, wherein said reheating index is obtained by parametric model fitting.

21. The method of claim 17, wherein said measured thermal response is analyzed numerically according to a layered bio-heat equation based on the following formula or variations thereof:
$${ρ}_{n} {C}_{n} \frac{\partial {T}_{n}}{\partial t} = {k}_{n} {∇}^{2} {T}_{n} + {ρ}_{b} {C}_{b} {w}_{b} ( {T}_{b} - {T}_{n} ) + {Q}_{met} ,$$
wherein n is the n^{th} layer, ρc is thermal capacity of tissue, T is spatial temperature distribution in tissue, k is thermal conductivity of tissue, ρ_{b}c_{b} is thermal capacity of blood, w_{b} is blood perfusion rate, T_{b} is spatial temperature distribution in blood, and Qₘₑₜ, is the metabolic heat generation per unit volume.

22. A computer readable medium, comprising software, which software, when executed by a computer, causes the computer to implement the method of 12.
